(19) United States
(12) Patent Application Publication
(10) Pub. No.: US 2025/0107881 A1
(43) Pub. Date: Apr. 3, 2025

(54) SYSTEM AND METHOD FOR PUMPING A FLUID THROUGH A TARGET UNIT

(71) Applicant: The University of Sydney, Camperdown (AU)

(72) Inventors: Steven Garry Wise, Camperdown (AU); Miguel Correia Dos Santos, Camperdown (AU); Timothy Charles Mitchell, Camperdown (AU)

(21) Appl. No.: 18/834,353

(22) PCT Filed: Feb. 1, 2023
(86) PCT No.: PCT/AU2023/050061

§ 371 Date: Jul. 30, 2024

(30) Foreign Application Priority Data
Feb. 1, 2022 (AU) 2022900186

(51) Int. Cl. A61F 2/06

(52) U.S. Cl. CPC A61F 2/06; A61F 2240/008

(57) ABSTRACT

The invention provides a system for pumping a fluid through a target unit, comprising a source of compressed gas, a gas circuit connected to said source of compressed gas and including gas flow control apparatus, a first connection and a second connection for connecting to, respectively, afferent and efferent ends of the target unit, a fluid flow circuit in fluid communication with said first and second connections, the fluid flow circuit including fluid control apparatus and a first and second fluid reservoir, wherein the gas flow control apparatus provides coordinated delivery and release of compressed gas to dynamically control fluid level in each of said first and second fluid reservoirs in order to provide, by way of operation of the fluid control apparatus in the fluid flow circuit, continuous fluid flow through the target unit in a single direction in accordance with a predetermined flow profile.

### CROSS-REFERENCE TO RELATED APPLICATIONS

The present patent application is a U.S. National Stage Application, filed under 35 U.S.C. § 371, of International Application No. PCT/AU2023/050061, filed Feb. 1, 2023, which claims the right of priority for the Australian patent application 2022900186, filed on Feb. 1, 2022, the content of which is incorporated herein by reference in their entireties.

### FIELD OF THE INVENTION

The invention provides a system and a method for pumping a fluid through a target unit. In particular, the invention provides a system and method for driving fluid flow through a bioreactor, such as a vascular or other tissue construct, in order to mimic physiological conditions.

### BACKGROUND OF THE INVENTION

There is a need for biocompatible materials to replace diseased autogenous vessels in patients suffering from cardiovascular disease. Synthetic vascular constructs developed in vitro are used to study biological signalling and to assess novel biomaterials and biomedical devices. However, traditional in vitro models have been generally unsatisfactory as they fail to replicate key pulsatile haemodynamic factors such as pressure, flow rate and shear stress.

Models based on bioreactor technology have attempted to incorporate pulsatile flow using positive displacement pumps such as peristaltic pumps, piston pumps and diaphragm pumps. However, accurate simulation of physiological waveforms requires independent control of pressure and flow over a wide range. Unfortunately, pressure and flow outputs are closely linked in such positive displacement pump systems.

This invention proposes an alternative system of driving pressure and flow that enables more accurate simulation of desired biological conditions.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

In accordance with a first aspect of the invention, there is provided a system for pumping a fluid through a target unit, comprising:
a source of compressed gas;
a gas circuit connected to said source of compressed gas and including gas flow control apparatus;
a first connection and a second connection for connecting to, respectively, afferent and efferent ends of the target unit;
a fluid flow circuit in fluid communication with said first and second connections, the fluid flow circuit including fluid control apparatus and a first and second fluid reservoir;
wherein the gas flow control apparatus provides coordinated delivery and release of compressed gas to dynamically control fluid level in each of said first and second fluid reservoirs in order to provide, by way of operation of the fluid control apparatus in the fluid flow circuit, continuous fluid flow through the target unit in a single direction in accordance with a predetermined flow profile.

The gas flow control apparatus therefore uses selective delivery and release of pressurised gas to provide coordinated inflation and deflation of the two fluid reservoirs, so to drive movement of the fluid. In particular, inflation is used by the fluid control apparatus to drive fluid flow in the fluid flow circuit to pump fluid to the first connection while deflation is used by the fluid control apparatus to allow fluid intake from the second connection. Each fluid reservoir therefore serves as a gas-driven fluid pump, the controlled application of the pressure of the compressed gas to the fluid in that reservoir acting to pump the fluid in a manner determined and regulated by the fluid control apparatus.

As will be understood, the emptying of one of the first and second reservoirs (ie. the ‘active’ reservoir), through delivery of compressed gas, results in movement of fluid to the other reservoir, thereby priming said other reservoir so that it can switch to becoming the ‘active’ reservoir without disruption of flow through the target unit.

It will be appreciated, reference to ‘continuous’ fluid flow refers to the provision of uninterrupted flow, as opposed to (for example) a constant rate of flow. The flow profile may involve, for example, a pulsatile fluid flow, a periodic fluid flow, or a fluid flow at a constant flow rate.

The target unit may be a component removably connectable to the other components of the system or may form an integral part of the system or a part of the system.

In a typical application, the target unit is a bioreactor (for example, a vascular graft), the system configured to provide or to simulate desired biological conditions therein.

The system of the invention is able, under dedicated control, to generate and modulate physiological vascular pressure, flow and shear conditions in a pulsatile flow profile in vascular grafts and similar and to allow real-time monitoring of these parameters, in a manner significantly more effective than the prior art. Tissue cells respond to these haemodynamic physiological cues, and achieving suitable conditions has been shown to provide clear benefits, including in terms of the alignment and functionality of the endothelial cells grown on grafts.

The gas flow control apparatus preferably comprises a valve arrangement governing provision of the compressed gas to each of the first and the second reservoirs. This valve arrangement may comprise, for example, a first proportional solenoid valve controlling connection from the source of compressed gas to the first reservoir and a second proportional solenoid valve controlling connection from the source of compressed gas to the second reservoir.

The gas circuit preferably includes an outlet gas flow apparatus to allow release of the compressed gas on deflation of either of the first and second fluid reservoirs. This may be provided by a pressure relief valve arrangement or a logic-controlled outflow mechanism. In one embodiment, a common outlet gas flow apparatus (such as a single pressure relief valve arrangement) may be operatively associated with both the first and the second fluid reservoirs. Alternatively, a separate outlet gas flow apparatus may be operatively associated with each of the respective first and second fluid reservoirs.

The predetermined flow profile is preferably a pulsatile fluid flow designed to mimic hydrodynamic fluid conditions in the vasculature of an animal body, preferably the human body. The target unit may therefore be a vascular construct, such as a 3D scaffold supporting a porous graft on which tissue cells have been grown, including vascular endothelial cells. The fluid may therefore be a biological fluid such as cell culture media, blood or a blood analog.

The hydrodynamic fluid conditions may include fluid pressure, flow and shear rate. The control of pressure at both ends of the target unit provides the ability to regulate these different fluid flow parameters, in contrast to prior art approaches that generally control only target unit inlet pressure. The fluid control apparatus, alone or in combination with the gas flow control apparatus, allows adjustment of flow and pressure through the target unit to compensate for varying resistance between vascular constructs with different diameters and geometry. Preferably, the gas flow control apparatus is configured to enable selective and independent adjustment of flow and pressure in the target unit.

In an embodiment, the gas flow control apparatus is configured to adjust a pressure differential between the first reservoir and the second reservoir.

In an embodiment, the gas flow control apparatus is configured to adjust the flow rate of the fluid flow through the target unit by adjusting the pressure differential between the first reservoir and the second reservoir.

In an embodiment, the gas flow control apparatus is configured to adjust a pressure at the target unit by adjusting pressure at the first reservoir and/or the second reservoir. In an embodiment, the pressure at the target unit is adjusted without substantially adjusting the flow rate of the fluid flow through the target unit. Thus, advantagously, a given flow rate through the target unit can be achieved whilst employing a different pressure condition in the target unit. In an embodiment, pressure in the target unit is approximately the average of the pressure of the first and second reservoirs. In an embodiment, the flow rate of the fluid flow through the target unit is adjusted without substantially adjusting the pressure in the target unit. Thus, advantagously, a given pressure in the target unit can be achieved whilst employing a different flow condition in the target unit.

The source of compressed gas may be a connection to a gas cylinder or similar. Further, the system may include a gas cylinder or similar, or may include a compressor to provide pressurised gas. The compressed gas may be air or any suitable gas or mix of gases, including an inert gas such as nitrogen. Preferably, the compressed gas is of a composition that enables exchange of oxygen and carbon dioxide with the fluid for cell culture.

The fluid flow circuit is preferably a two-phase recirculation system by which the first connection and the second connection are in fluid communication with the first and second fluid reservoirs under control of said fluid control apparatus. It will be appreciated that said control can be passive or active control, depending on the components used in the fluid control circuit.

The fluid control apparatus preferably comprises a plurality of one-way valves configured to allow two modes of fluid flow, being (a) driving fluid from the first fluid reservoir to said first connection and from said second connection to the second fluid reservoir, and (b) driving fluid from the second fluid reservoir to said first connection and from said second connection to the first fluid reservoir, alternation between modes (a) and (b) providing the desired fluid flow profile. The plurality of one-way valves may be provided by a passive flow control arrangement, in which the one-way valves are simple check valves arranged to cause or permit the desired mode of fluid flow in accordance with the operation of the first and second fluid reservoir. Alternatively, the plurality of one-way valves may be provided by an active flow control arrangement, in which the one-way valves are actively controllable to cause or permit the desired mode of fluid flow.

Preferably, coordinated operation of the gas flow control apparatus and the fluid control apparatus enables switching between the two modes of fluid flow. In one embodiment, said coordinated operation of the gas flow control apparatus and the fluid control apparatus enables continuous alteration between the two modes of fluid flow.

The gas flow control apparatus may be controlled by a central logic controller, programmed to provide the determined fluid flow profile. Control of the gas flow control apparatus enables corresponding control of the fluid control apparatus. In an alternative embodiment, both the gas flow control apparatus and the fluid control apparatus may be controlled by the central logic controller. As will be understood, the outlet gas flow apparatus may also be controlled by the central logic controller.

The system may include the target unit itself, and/or a support for the target unit, such as a housing comprising the afferent port and efferent port. In an embodiment, the housing may define a chamber containing the target unit, in which conditions of the interior chamber may be controlled in order to mimic physiological conditions external to the target unit.

The source of compressed gas may comprise a suitable connection to allow coupling to a gas cylinder or compressor, or may include a gas cylinder or compressor.

It will be appreciated that whilst the system is largely described for pumping a fluid through a bioreactor, the system can also be utilised in other applications that would benefit from more accurate generation and/or modulation of pressure, flow and/or shear conditions and allow real-time monitoring of these parameters.

As will be understood, the system may include more than two reservoirs and/or multiple fluid flow circuits, for example to provide more complex fluid flow profiles.

In accordance with a second aspect of the invention, there is provided a method for pumping a fluid through a target unit in a fluid flow circuit, the fluid flow circuit including two fluid reservoirs and a fluid control apparatus, the method comprising operating gas flow control apparatus to control supply of compressed gas to dynamically control filling and discharge of a first and a second fluid reservoir and, in a manner coordinated with the operation of the gas flow control apparatus, using the fluid control apparatus in the fluid flow circuit to provide fluid flow through the target unit in a single direction in accordance with a predetermined flow profile.

Preferably, the method includes using the fluid control apparatus to alternate connection between two fluid flow routes, namely: (a) from the first fluid reservoir to an afferent end of the target unit and from an efferent end of the target unit to the second fluid reservoir: and (b) from the second fluid reservoir to an afferent end of the target unit and from an efferent end of the target unit to the first fluid reservoir, the alternation between routes (a) and (b) being conducted in accordance with operation of the gas flow control apparatus controlling the operation of the first and second fluid flow reservoirs.

It will be appreciated that features disclosed with respect to the first aspect of the invention are also applicable with respect to the second aspect of the invention described above, including different combinations of features disclosed.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1 is a schematic diagram of a bioreactor system in accordance with an embodiment of the invention;

FIG. 2 is a schematic diagram of a reservoir, with associated gas and fluid flow ports, of the bioreactor system of FIG. 1;

FIG. 3A is a schematic diagram of a fluid flow circuit of the bioreactor system of FIG. 1, with fluid flowing along a first pathway;

FIG. 3B is a schematic diagram of the fluid flow circuit of the bioreactor system of FIG. 1, with fluid flowing along a second pathway;

FIG. 4A is a graph showing control of frequency of pulsatile pressure over a physiological range of 30-120 bpm by manipulation of the opening frequency of proportional valves;

FIG. 4B is a graph showing control of pulse width by manipulation of the duty cycle of proportional valves;

FIG. 4C is a graph showing control of pressure offset by manipulation of the relief valve set pressure;

FIG. 4D is a graph showing control of pressure pulse amplitude by controlling the compressed gas pressure at the inlet of the proportional valves;

FIG. 4E is a graph showing control of pressure pulse amplitude after adjusting the pressure offset;

FIGS. 5A and 5B are graphs showing the difference in the smoothness of the waveforms produced using an instantaneous valve relative to a proportional valve, and FIG. 5C shows the flow data corresponding to the pressure waveform of FIG. 5B;

FIG. 6 illustrates controllable valve parameters for defining the opening and closing behaviour of the proportional valves;

FIG. 7 is a graph illustrating the relationship between fluid reservoir pressure differential and flow rate;

FIG. 8 is a graph illustrating pressure at the vascular construct verses flow rate;

FIG. 9A shows reservoirs suitable for use in the bioreactor system of FIG. 1;

FIG. 9B is a side cross-sectional view of the reservoir shown in FIG. 9A; and

FIG. 9C is a cross-sectional perspective partial view of the reservoir shown in FIG. 9A, which shows the internal form.

### DETAILED DESCRIPTION OF THE EMBODIMENTS

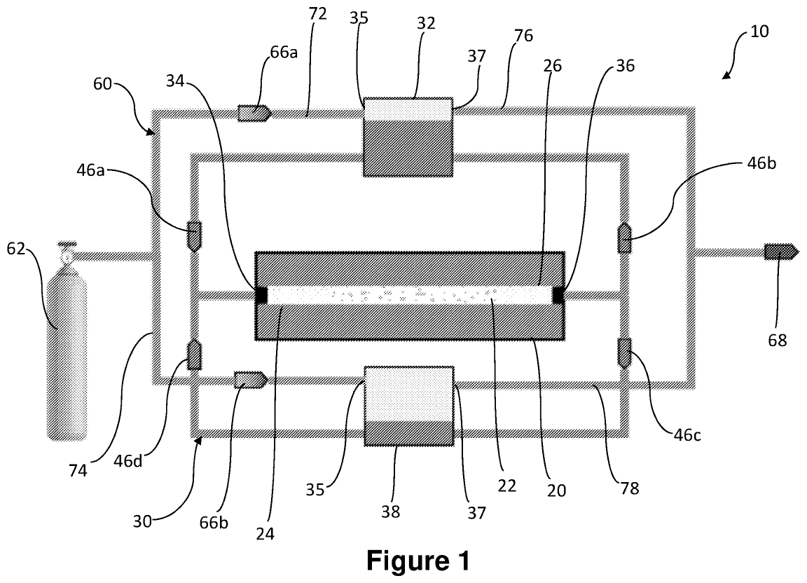

Reference is now made to FIG. 1, which illustrates a bioreactor system 10 in accordance with an embodiment of the invention. Bioreactor system 10 includes a bioreactor chamber 20, in which is housed a synthetic vascular construct 22. In this embodiment, vascular construct 22 is a 3-dimensional scaffold supporting a porous graft on which tissue cells are being or have been grown, including vascular endothelial cells. Bioreactor system 10 has therefore been designed to mimic hydrodynamic fluid conditions in the vasculature of an animal body in vitro. It will be appreciated that in other embodiments, chamber 20 may house constructs intended to mimic fluid conditions for other biological processes of an animal body.

As will be understood, the system can be used in tissue engineering, ie. in the development and manipulation of laboratory-grown molecules, cells, tissues or organs to replace or support the function of defective or injured body parts, including growing complex, three dimensional tissues.

Vascular construct 22 is secured in chamber 20 so as to extend between two opposing ends thereof. Vascular construct 22 includes an afferent end 24 adjacent a first end of the chamber 20, and an efferent end 26 adjacent a second end of chamber 20. Vascular construct 22 is in fluid communication with a fluid flow circuit 30 via the chamber ports at afferent and efferent ends 24, 26. In particular, fluid flow circuit 30 is in fluid communication with vascular construct 22 via a first connection 34 connected to the afferent end 24 and a second connection 36 connected to the efferent end 26 of the vascular construct 22 respectively. Fluid flow circuit 30 is configured, during operation, to provide continuous fluid flow through the vascular construct 22 in a single direction in accordance with a predetermined flow profile, as will be explained further below.

Fluid flow circuit 30 includes a first fluid reservoir 32 and a second fluid reservoir 38, each in fluid communication with vascular construct 22. Fluid reservoirs 32, 38 are configured to contain a biological fluid, being a liquid such as a cell culture media, blood or a blood analog (eg. a suitable media with an additive to provide the unique fluid properties of blood), which is circulated around fluid flow circuit 30, between the first fluid reservoir 32 and second fluid reservoir 38.

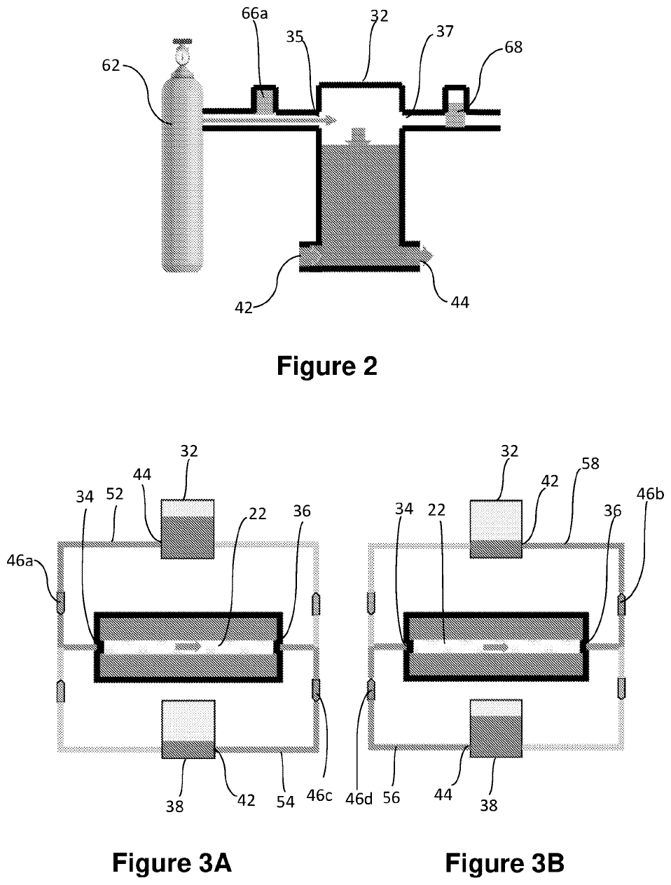

With reference to FIG. 2, which provides a schematic diagram of fluid reservoir 32 (fluid reservoir 38 is of substantially the same construction), fluid reservoir 32 includes a fluid inlet port 42 and an opposed fluid outlet port 44 each disposed at a lower end of reservoir 32. In operation, fluid travels into and out of fluid reservoir 32 via the inlet port 42 and outlet port 44 respectively. Fluid reservoir 32 further includes a gas inlet port 35 and an opposing gas outlet port 37, each of port 35 and 37 disposed at an upper end of reservoir 32. Reservoirs 32, 38 are in fluid communication with a gas circuit 60 (FIG. 1) via ports 35 and 37, whereby coordinated delivery and release of gas into reservoirs 32, 38 drive the movement of fluid about flow circuit 30. Fluid reservoir 32 may be of an alternative construction to that depicted in FIG. 2 in order to assist in optimal functioning. For example, the location of the fluid and gas ports may differ to that shown, e.g. fluid inlet port 42 may be located towards a lower portion of reservoir 32.

Returning to FIG. 1, flow circuit 30 includes a plurality of one-way check valves 46a-d (four in the present embodiment). Check valves 46a-d are disposed along flow circuit 30 as shown and serve to passively control the flow of fluid therethrough. Check valves 46a-d are passively resealing one-way valves with a fixed cracking pressure, such as Qosina Medical check valves (manufacturer part number (MPN) 80503). However, in alternative embodiments, valves 46a-d may be actively controllable by a suitable controller in operative communication with check valves 46a-d to electronically control the valve. Sensors (not shown) may be disposed along flow circuit 30 for detecting characteristics of the fluid (e.g. pressure, flow rate, etc) in the fluid flow circuit 30. Information monitored by the sensors can be used to control operation or selection of check valves 46a-d (eg. in a feedback loop system, by operator control, and/or adjustment of the cracking pressure).

Check valves 46a-d are disposed along fluid flow circuit 30 to allow for two modes of fluid flow. A first fluid mode involves the fluid being driven along a first fluid pathway, best shown in FIG. 3A, from first reservoir 32 to second reservoir 38. The first fluid pathway is defined by: a first outflow conduit 52 extending between reservoir 32 and first connection 34; check valve 46a enabling fluid to flow along first outflow conduit 52 from reservoir 32 to first connection 34, but prevent flow of fluid in the reverse direction; vascular construct 22; a first inflow conduit 54 extending between second connection 36 and reservoir 38; and check valve 46c enabling fluid to flow along first inflow conduit 54 from second connection 36 to reservoir 38, but prevent flow of fluid in the reverse direction. First outflow conduit 52 is connected at an upstream end thereof to outlet port 44 of first reservoir 32 and is connected at a downstream end thereof to first connection 34. First inflow conduit 54 is connected at an upstream end thereof to second connection 36 and connected at a downstream end thereof to inlet port 42 of second reservoir 38.

A second fluid mode involves the fluid being driven along a second fluid pathway, best shown in FIG. 3B, from second reservoir 38 to first reservoir 32. The second fluid pathway is defined by: a second outflow conduit 56 extending between second reservoir 38 and first connection 34; check valve 46d enabling fluid to flow along second outflow conduit 56 from reservoir 38 to first connection 34, but prevent flow of fluid in the reverse direction; vascular construct 22; a second inflow conduit 58 extending between second connection 36 and first reservoir 32; and check valve 46b enabling fluid to flow along second inflow conduit 58 from second connection 36 to first reservoir 32, but prevent flow of fluid in the reverse direction. Second outflow conduit 56 is connected at an upstream end thereof to outlet port 44 of second reservoir 38 and connected at a downstream end thereof to first connection 34. Second inflow conduit 58 is connected at an upstream end thereof to second connection 36 and connected at a downstream end thereof to inlet port 42 of first reservoir 32.

With reference again to FIG. 1, gas circuit 60 is connected to a gas source in the form of gas cylinder 62. Compressed gas from gas cylinder 62 is used to drive the fluid contained in fluid reservoirs 32, 38 about fluid flow circuit 30. In the present embodiment, the compressed gas comprises a gas mixture consisting of about 83% N₂, 12% O₂, and 5% CO₂. Such a composition provides a suitable exchange of oxygen and carbon dioxide for cell culture inside chamber 20. In this way, the compressed gas circulated about gas circuit 60 has dual functions—driving movement of fluid about fluid flow circuit 30, and providing suitable gas exchange between the gas and the liquid fluid. In some embodiments, it will be desirable to adjust the gas mixture according to pH and oxygen requirements for suitable cell culture. In one example, it has been found that cell medium pH in the fluid is stable at 7.4 when regulated by CO₂ exchange within the reservoirs 32, 38.

Gas circuit 60 includes a pair of valves 66a,b disposed therealong that are controllable by a gas control apparatus (not shown) to govern provision of the compressed gas to each of fluid reservoirs 32, 38. The gas control apparatus can include any suitable controller in operative communication with valves 66a,b. In an embodiment, a single logic controller can be used to provide both gas and fluid control when actively controllable valves are used in the fluid flow circuit 30. The gas control apparatus may also include sensors (not shown), disposed along gas circuit 60, configured to detect characteristics of the gas (e.g. pressure, flow rate, etc). Data from the sensors can be used in controlling operation of valves 66a,b.

Valve 66a is a proportional solenoid valve controlling the flow of compressed gas from gas cylinder 62 to first reservoir 32, and valve 66b, also in the form of a proportional solenoid valve, controls the flow of compressed gas from gas cylinder 62 to second reservoir 38. As will be understood, a proportional valve of this sort allows precise modulation of gas flow (ie. infinitely variable positioning between 0 and 100% of opening). An example of a suitable proportional valve is a Burkert Type 2861 (MPN: 249897). Gas circuit 60 includes a first gas inflow conduit 72 extending between gas cylinder 62 and first reservoir 32, wherein proportional valve 66a is disposed along conduit 72 for controlling gas flow from gas cylinder 62 to fluid reservoir 32. Gas inflow conduit 72 is connected at a downstream end thereof to gas inlet port 35 of reservoir 32 and is fluidly connected at an upstream end thereof to gas cylinder 62. In the present embodiment, said upstream connection of gas inflow conduit 72 is connected to one of the downstream ends of a first T-connector (not shown), the upstream end of the first T-connector fluidly connected to gas cylinder 62.

Gas circuit 60 further includes a second gas inflow conduit 74 extending between gas cylinder 62 and second reservoir 38, wherein proportional valve 66b is disposed along conduit 74 for controlling gas flow from gas cylinder 62 to second reservoir 38. Gas inflow conduit 74 is connected at a downstream end thereof to gas inlet port 35 of reservoir 38 and is fluidly connected at an upstream end thereof to gas cylinder 62. In the present embodiment, said upstream connection of gas inflow conduit 74 is connected to the other downstream end of the first T-connector.

Gas circuit 60 further includes a first gas outflow conduit 76 extending between first reservoir 32 and a pressure relief valve 68. Pressure relief valve 68 is configured to exhaust any excess or undesired pressure in gas circuit 60. Pressure relief valve 68 can be either a passive spring valve with an adjustable cracking pressure, or an electronically controlled valve. As will be appreciated from the description below, in embodiments where an electronically controlled pressure relief valve is used, pressure control of gas circuit 60 can be achieved at least in part through control of the pressure relief valve, ie. pressure control of gas circuit 60 can be controlled at a downstream end of gas circuit 60. A suitable electronically controlled pressure relief valve includes a Burkert Type 2861 (MPN: 249897), and a suitable passive spring valve includes a Kegland Blowtie Spunding Valve (MPN: KL09706).

Outflow conduit 76 is connected at an upstream end thereof to gas outlet port 37 of first reservoir 32, and is fluidly connected at a downstream end thereof to pressure relief valve 68. In the present embodiment, said downstream connection of gas outflow conduit 76 is connected to one of the upstream ends of a second T-connector (not shown), the downstream end of the second T-connector fluidly connected to pressure relief valve 68. Gas circuit 60 further includes a second gas outflow conduit 78 extending between second reservoir 38 and pressure relief valve 68. Outflow conduit 78 is connected at an upstream end thereof to gas outlet port 37 of second reservoir 38, and is fluidly connected at a downstream end thereof to pressure relief valve 68. In the present embodiment, said downstream connection of gas outflow conduit 78 is connected to the other upstream end of the second T-connector. Whilst the depicted embodiment provides a single pressure relief valve 68 in gas circuit 60, it will be appreciated that in alternative embodiments, separate pressure relief valves can be provided in fluid communication with first reservoir 32 and second reservoir 38 respectively. As will be understood, providing separate pressure relief valves in fluid communication with respective first and second reservoirs 32, 38 enables independent control of the pressure in each reservoir, and thus a greater degree of control of the operation of the system.

Operation of gas circuit 60 to drive the flow of fluid about fluid flow circuit 30 will now be described. Gas cylinder 62 is opened to enable the flow of compressed gas from gas cylinder 62 about gas circuit 60. Valves 66a,b govern the flow of compressed gas that enters the respective reservoirs 32, 38. It will be understood that pressure is controlled within the reservoirs 32, 38 by controlling the pressure of the gas above the fluid, i.e. the gas entering/exiting the reservoirs 32, 38 via gas inlet/outlet ports 35, 37. The gas flow control apparatus enables selective delivery and release of pressurised gas to and from the reservoirs 32, 38 to provide coordinated inflation and deflation of fluid reservoirs 32, 38, and therefore drive movement of the fluid about fluid flow circuit 30. The gas flow control apparatus enables this selective delivery and release by controlling the opening and closing of valves 66a,b and relief valve 68.

For example, in order to drive fluid from the first reservoir 32 to the second reservoir 38 (along the first fluid pathway), first reservoir 32 is inflated, i.e. pressurised, by controlled delivery of compressed gas therein. This is achieved by opening valve 66a to a desired degree, whilst valve 66b is closed. This causes a pressure differential between the upstream end of the vascular construct 22 (at the high pressure first reservoir 32) and the downstream end of the vascular construct (at the relatively low pressure second reservoir 38), thereby driving fluid about the fluid flow circuit 30 along the first fluid pathway. This results in the fluid being pumped to vascular construct 22 through first connection 34 and pumped away from vascular construct 22 through second connection 36 to the second reservoir 38. In conjunction with the inflation of first reservoir 32 in this example, deflation of second reservoir 38 by controlled release of compressed gas therefrom can also take place to increase the pressure differential between the high pressure first reservoir 32 and the low pressure second reservoir 38. This assists in driving fluid towards the second reservoir 38 along the first fluid pathway. This allows the fluid to therefore be pumped away from vascular construct 22 through second connection 36 to second reservoir 38.

Valve 66b can then be opened to a desired degree, whilst valve 66a is closed. This results in the reverse operation (i.e. inflation of the second reservoir 38 and deflation of the first reservoir 32). In the embodiment illustrated, alternating between movement of the fluid along the first fluid pathway and the second fluid pathway is undertaken continuously in accordance with a relatively high frequency pulse of operation of valves 66a,b. However, this alternation between movement of the fluid along the first and second fluid pathways can be undertaken less frequently or be dependent on other factors. For example, determination as to when to switch direction of fluid flow can be determined based on the measured fluid level in a respective reservoir. For example, once the fluid level in a receiving reservoir reaches a prescribed level, operation of valves 66a, b may be switched to allow the flow of fluid to begin travelling along the other fluid pathway. Pressure sensors or fluid level sensors can be used to determine this switching threshold.

Each fluid reservoir 32, 38 therefore serves as a gas-driven fluid pump, the controlled application of the pressure of the compressed gas to the fluid in the respective reservoir acting to pump the fluid in a manner determined and regulated by the fluid control apparatus. The two modes of fluid flow therefore define a ‘figure of 8’ circuit, whereby check valves 46a-d maintain consistent flow through vascular construct 22 in a single direction. It will be appreciated that bioreactor system 10 therefore enables independent control of the pressure and flow rate (and as a consequence, the shear stresses) experienced by vascular construct 22. This is because system 10 enables not only the control of absolute pressure in the system 10, but the control of pressure at both the afferent end 24 and efferent end 26 of the vascular construct 22, this control of the pressure gradient or pressure differential between the afferent end 24 and efferent end 26 of the vascular construct 22 being proportional to controlling the flow rate through the vascular construct 22. Bioreactor system 10 therefore enables the decoupling of fluid flow control and pressure control, i.e. independent control of each parameter, which through the fluid and gas control apparatus enable accurate simulation of pulsatile flow conditions.

In operation, the fluid is circulated about fluid flow circuit 30, between the first fluid reservoir 32 and second fluid reservoir 38 via either the first or second fluid pathway depending on the operation of the gas circuit 60. It will be appreciated that the proportional valves 66a,b enable adjustment of flow and pressure through the vascular construct 22 to compensate for varying resistance between vascular constructs with different diameters and geometry. This enables continuous fluid flow through the vascular construct 22 in a single direction in accordance with a predetermined flow profile (e.g. desired pulsatile flow conditions). The fluid flow circuit 30 is therefore a two-phase recirculation system by which the first connection 34 and the second connection 36 are in fluid communication with the first and second fluid reservoirs 32, 38 under control of the fluid control apparatus. This two-phase recirculation system avoids the need for a filling stage to replenish the fluid in the system. Instead, the same fluid is continuously circulated in the fluid flow circuit 30 and there is no disruption to the flow conditions experienced by the vascular construct 22 as the direction of fluid passage therethrough remains in the same direction. In other words, the flow stimulation through vascular construct 22 is effectively identical in both phases (i.e. irrespective of whether the fluid is flowing along the first or second pathways).

Reference again is made to FIG. 1 and FIG. 3A, the latter of which provides a schematic representation of fluid flowing along the first fluid pathway. In the state shown in FIG. 3A, the fluid level in reservoir 32 is greater than the fluid level in reservoir 38. In order for fluid to flow along the first fluid pathway, first reservoir 32 assumes the role of the high pressure reservoir, whilst second reservoir 38 assumes the role of the low pressure reservoir. This results in the movement of fluid along the first fluid pathway (i.e. the movement of fluid from the first reservoir 32 to the second reservoir 38). Valve 66a in gas circuit 60 is controlled to provide a desired flow of compressed gas into reservoir 32 through gas inlet port 35. For the purposes of this example, relief valve 68 is closed at this stage (resulting in minimal flow of gas out of gas outlet port 37), thereby enabling the pressure within reservoir 32 to increase. This pressure build up caused by the compressed gas entering reservoir 32 imparts a downward force onto the fluid contained in reservoir 32, thereby forcing the fluid out of reservoir 32 from fluid outlet port 44, along outflow conduit 52 and through afferent end 24 of vascular construct 22 via first connection 34. The fluid then travels out the efferent end 26 of vascular construct 22 via second connection 36, along inflow conduit 54 and into reservoir 38 via fluid inlet port 42. It will be appreciated that in this mode of operation, reservoir 38 is operating under a low pressure condition, with valve 66b closed or substantially closed to prevent gas induced pressure build up in reservoir 38.

In order to maintain continuous fluid flow through vascular construct 22, the flow of fluid must be reversed so that the fluid travels from the second reservoir 38 back to the first reservoir 32. However, to provide the required one-way flow of fluid through vascular construct 22, flow of fluid is redirected so that rather than travelling back along the first fluid pathway, the fluid is made to move along the second fluid pathway as shown in FIG. 3B (the abovementioned “figure of 8” circuit). Valve 66b is controlled to allow the delivery of compressed gas into reservoir 38 through gas inlet port 35. Once again, relief valve 68 is closed at this stage, thereby enabling the pressure within reservoir 38 to increase. This pressure build up caused by the compressed gas entering reservoir 38 imparts a downward force onto the fluid contained in reservoir 38, thereby forcing the fluid out of reservoir 38 from fluid outlet port 44, along outflow conduit 56 and through afferent end 24 of vascular construct 22 via first connection 34. The fluid then travels out the efferent end 26 of vascular construct 22 via connection 36, along inflow conduit 58 and into reservoir 32 via fluid inlet port 42. It will be appreciated that in this mode of operation, reservoir 32 is operating under a low pressure condition, with valve 66a closed or substantially closed to prevent gas induced pressure build up in reservoir 32.

The above passages describe the general operation of bioreactor system 10, namely how the compressed gas in gas circuit 60 is used to drive the movement of fluid about the fluid flow circuit 30. In order to utilise this process to mimic pulsatile flow conditions at vascular construct 22, the functioning of the gas flow control apparatus and fluid control apparatus will now be described, including describing how gas circuit 60 enables selective and independent adjustment of flow and pressure in vascular construct 22. Coordination of inflation and deflation of reservoirs 32, 38 enables pulsatile flow adjustable pulsatile conditions. As will be understood, the particular flow paths are enabled by the ‘figure of 8’ flow circuit topology (FIGS. 3A and 3B).

Various sensors (not shown) can be utilised in system 10 to ensure desired operation thereof. These can include:
Pressure sensors can be provided at each of first reservoir 32 and second reservoir 38 to measure the pressure therein. These pressure sensors can be provided as inline sensors or integrated within the reservoir itself. An example of a suitable pressure sensor is the Pendotech Preps-N-012.
Pressure sensor(s) can be provided at the vascular construct 22 at either or both of the afferent and efferent ends 24, 26 thereof to measure the upstream and/or downstream pressure. The inventors' preliminary observations suggest that the relationship between reservoir pressure and pressure at the vascular construct 22 may be stable enough that the pressure at the vascular construct 22 can be reliably and accurately inferred from the pressure in the reservoirs. Therefore, in a simplified form, pressure sensor(s) at the vascular construct 22 may be omitted from system 10.
Flow sensor(s) at either or both of the afferent and efferent ends 24, 26 of the vascular construct 22 can be provided to measure the flow rate of the fluid through vascular construct 22. An example of a suitable flow sensor is the Transonic ME3PXL.
Gas source pressure sensor can be provided at gas cylinder 62 to detect leaks, cylinder depletion or to provide information to the controller to compensate for various alternate gas sources.
Gas source mass flow sensor can be provided at the outlet of gas cylinder 62 to monitor gas consumption.
Chamber pressure sensor for measuring the pressure in chamber 20. In an embodiment, the pressure in chamber 20 can be controlled in a similar manner to the reservoirs.
Reservoir level sensors for monitoring the level of fluid in the reservoirs 32, 38 and/or chamber 20. These sensors could be used to assist in setting up system 10 (e.g. ensuring the correct starting volume in each reservoir), to automatically detect leaks, to monitor fluid evaporation or to provide data for automated waste removal and replenishment systems, and/or to determine the point of switching between the fluid flowing along the first or second fluid pathway. It is also envisaged that sufficiently accurate level sensors with a high sample rate could also be used as a unique means of flow sensing. This possibility is enabled by the unique behaviour of the two reservoirs in the fluid flow circuit 30. In a conventional flow loop, reservoir level stays constant (flow out=flow in) but in system 10 each reservoir is always in either an increasing or decreasing phase. Any change in reservoir level corresponds to the flow through the vascular construct in the same period. Rate-of-change of level can be cross-referenced at both reservoirs simultaneously for more accurate flow readings. An example of suitable level sensors include capacitive or camera-based sensors.
Sensors to monitor aspects of the fluid, such as a cell culture media, to ensure the conditions are suitable for cells and to determine when the media needs to be replaced. Suitable oxygen and pH sensors can be attached to the reservoirs or conduits of the fluid flow circuit 30. Output from these sensors may be sent to the controller for varying the composition of the gas to monitor and regulate dissolved oxygen and pH in the media. The output may also be used to indicate when removal of media waste is required and when replenishment of media is required. Sensors for monitoring levels of additional analytes, such as lactate, glucose, specific growth factors or drugs may also be used. An example of suitable sensors include optical sensors, such as a PreSens Sensor Stick.

Output of the sensors used in system 10 may also be used to provide feedback assisted control of the operation of the valves. Measured output can be compared to desired output, with valve control parameters adjusted accordingly (eg. using a suitable PID algorithm).

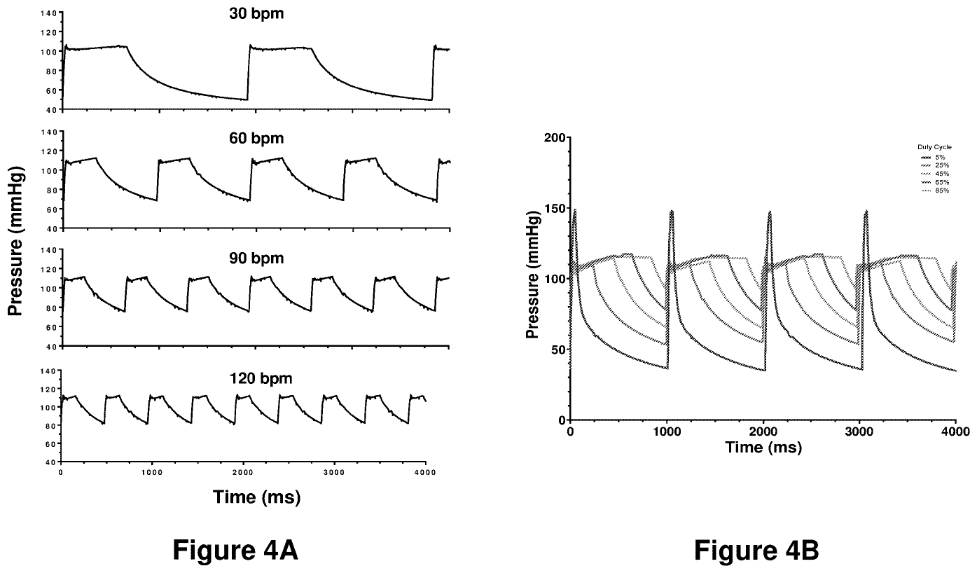

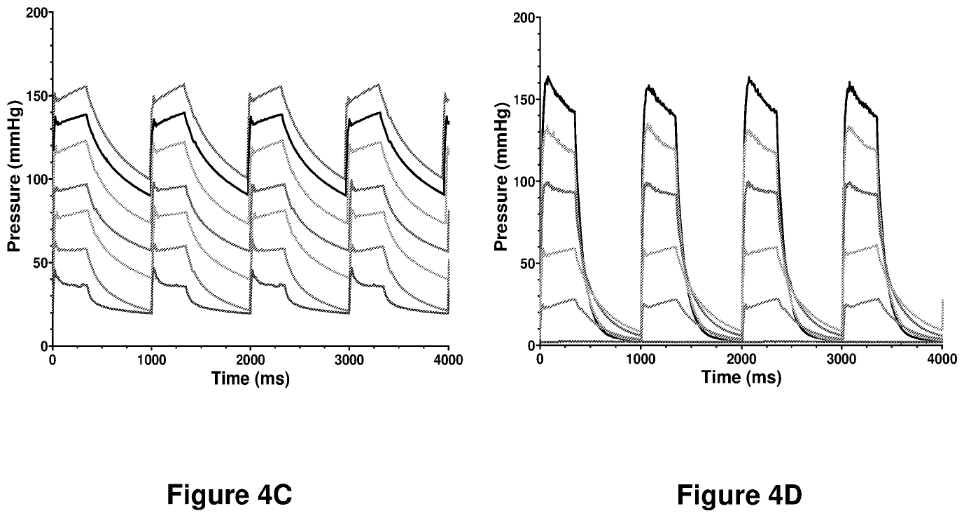

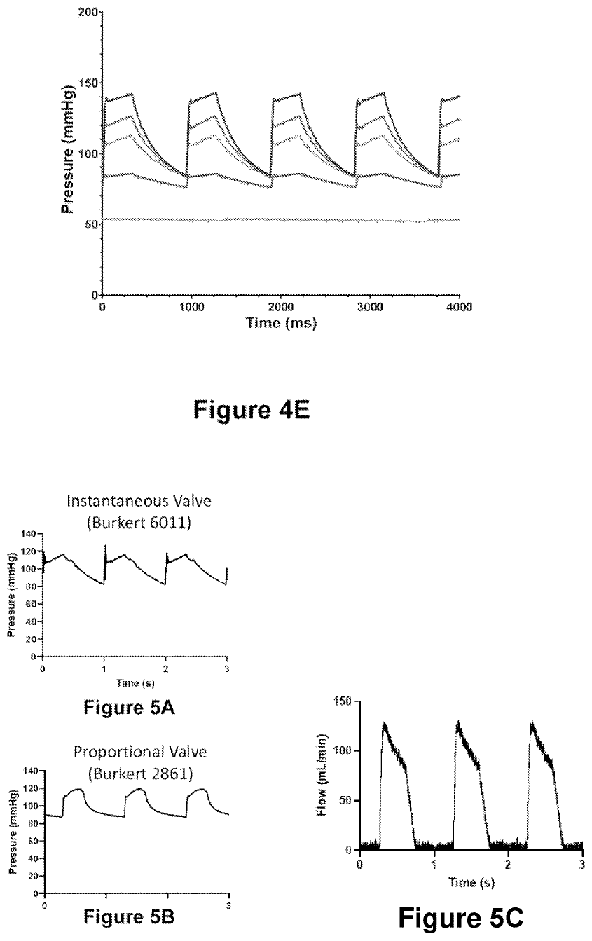

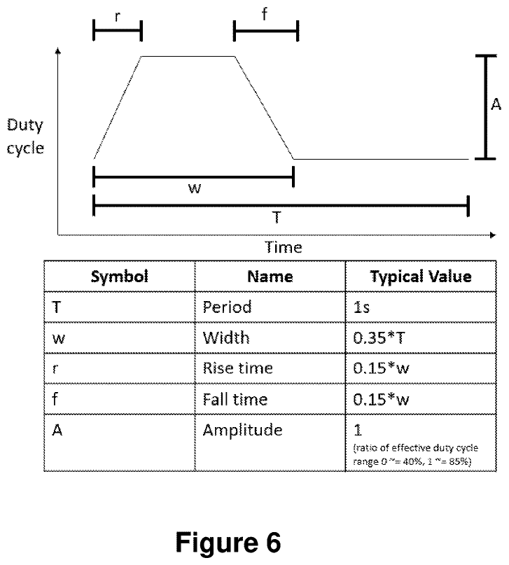

Through trials conducted by the inventors, pressure and flow waveforms have been produced with adjustable frequency (FIG. 4A), pulse width (FIG. 4B), offset (FIG. 4C), and amplitude (FIG. 4D). Coordination of pressure parameters has generated pressure and flow waveforms within the physiological range. FIG. 4A shows control of the frequency of pulsatile pressure over a physiological range of 30-120 bpm by manipulation of the opening frequency of proportional valves 66a,b. FIG. 4B shows control of pulse width by manipulation of the duty cycle of proportional valves 66a,b. FIG. 4C shows control of pressure offset by manipulation of the relief valve set pressure. FIG. 4D shows control of pressure pulse amplitude by controlling the compressed gas pressure at the inlet of the proportional valves 66a,b. FIG. 4E shows control of pressure pulse amplitude after adjusting the pressure offset. It will be evident that the use of proportional valves 66a,b, which can be set to open partially or to open gradually, allows for greater control of the waveform shape. This greater control is illustrated when comparing FIG. 5A and FIG. 5B, which shows the difference in the smoothness of the waveforms produced using an instantaneous valve relative to a proportional valve. FIG. 5C shows the flow data corresponding to the pressure waveform of FIG. 5B. FIG. 6 illustrates the controllable valve parameters for defining the opening and closing behaviour of the proportional valves 66a,b. It will be appreciated that this is just one example of a suitable control input, and that other control inputs may be used.

System 10 is capable of achieving a wide variety of flow profiles by adjusting various pressure and flow parameters within the system based on a predetermined or desired flow profile, e.g. to mimic hydrodynamic fluid conditions. For example, system 10 can achieve the following range of parameters: pulse rate between about 1-300 Hz; pressure (both systolic and diastolic) between 0-300 mmHg; flow rate between 0-500 ml/min (bearing in mind that greater flow rates can be achieved with larger vascular constructs and/or reservoirs than those employed in system 10). The shear stress on the vascular construct 22 can be calculated based in part on the measured parameters (such as flow rate), viscosity of the fluid media and the diameter of the vascular construct 22.

Information gathered from any of the sensors in bioreactor system 10 or graphical representations as shown in FIGS. 4A-E, FIGS. 5A-C and FIG. 6 can be displayed on a screen for visual inspection by an operator of system 10. A user interface may also be provided, enabling the operator to make desired operational changes in real-time.

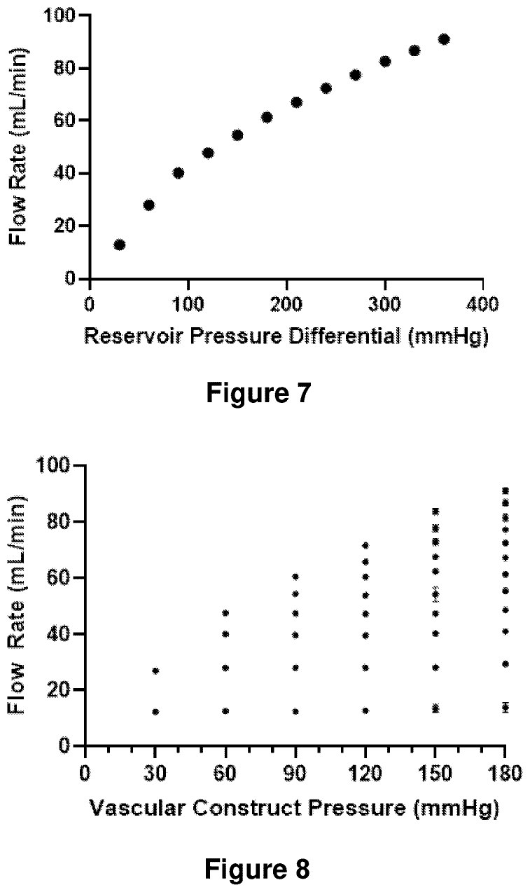

FIG. 7, which illustrates a relationship of the pressure difference between first fluid reservoir 32 and second fluid reservoir 38, demonstrates the desired pressure-controlled flow modulation, i.e. how controlling the pressure at fluid reservoirs 32, 38 can act to control flow rate through vascular construct 22.

FIG. 8, which illustrates pressure in vascular construct 22 against flow rate, demonstrates that various flow rates can be produced through vascular construct 22 for a given pressure at the vascular construct. For example, the flow rate through vascular construct 22 can be increased while maintaining pressure in the construct by increasing the pressure of one of the first and second reservoirs and decreasing the pressure of the other accordingly. The flow paths of fluid flow circuit 30 corresponding to the two modes described above are symmetrical, and therefore pressure in the centre of vascular construct 22 is approximately the average of the reservoir pressures. An example of two conditions with the same pressure and different flow rate is given below:
(1) First reservoir: 140 mmHG, second reservoir 100 mmHG->Construct 120 mmHG, 22 ml/min;
(2) First reservoir: 240 mmHG, second reservoir 0 mmHG->Construct 120 mmHG, 72 ml/min.

In the example of FIG. 8, the x-axis is limited by the set pressure of the gas cylinder regulator, whilst the y-axis is limited by the resistance of the fluid path between the fluid reservoirs and vascular construct, vascular construct geometry and fluid reservoir pressure (the fluid reservoirs have a minimum pressure equal to atmosphere).

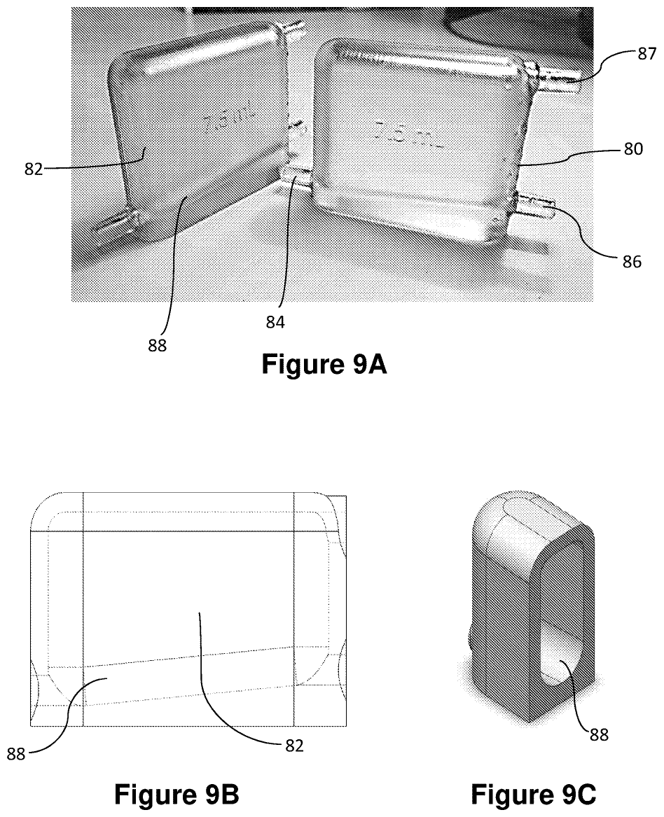

Reference is made to FIGS. 9A-9C, which illustrate one embodiment of a fluid reservoir 80 suitable for use in bioreactor system 10 (two reservoirs 80 are depicted in FIG. 7A). Reservoir 80 was produced using resin 3D printing (Formlabs Biomed Clear Resin, MPN:RS-F2-BMCL-01), although it will be appreciated that reservoir 80 can be produced from other materials suitable to ensure reservoir 80 functions as desired, including being a material that prevents toxicity of cells in the fluid media upon contact. Reservoir 80 defines an inner volume 82 having a concave shaped base 88 inclined from a fluid inlet port 84 to a fluid outlet port 86 (shown in FIGS. 9A and 9B).

The concave shaping of base 88 assists in reducing areas of turbulence and pockets of flow stagnation within reservoir 80 (which might otherwise allow attachment of cells to the reservoir base). The inclined base is designed to prevent entrapment of cells in the fluid and reservoir 80 through gentle expulsion of the cells. The incline of base 88 allows gravity driven “funnelling” of cells to fluid outlet port 86. It has been found that a slight incline of up to about 10° is sufficient to prevent cells from being trapped on the base 88 of the reservoir 80. Reservoir 80 can be customised in other ways to enable suitable function in bioreactor system 10. This includes the provision of a gas port 87 for connection to gas circuit 60.

As the figures show, only a single gas port 87 need be provided, which acts as both the inlet and outlet port for gas into reservoir 80. Use of a single port reduces the number of assembly points that must be kept sterile to avoid contaminating the gas. This is achieved by using a sterile syringe filter (not shown), one side of which is attached to gas port 87, the other side attached to a T or Y connector that branches to connect with the upstream and downstream conduits (respectively connecting to proportional valve 66a or 66b and pressure relief valve 68). A suitable syringe filter includes a Millex-GP syringe filter unit (MPN: SLGP033RS).

A further customisation of reservoir 80 is that of the inner volume level thereof to ensure suitable function of the fluid flow circuit 30. It is noted that inner volume 82 of reservoir 80 does not include a diaphragm or membrane separating the liquid fluid phase and the gas phase. This allows direct exchange of gases between the phases, the efficacy of which is enhanced by the increased gas pressure within the reservoir 80 during operation of the bioreactor system 10. It will be appreciated that in an alternative embodiment, a diaphragm or membrane may be provided in reservoir 80 to separate the liquid and gas phases. This has the benefit of less onerous sterility maintenance, as the membranes will reduce the possibility of contaminating the fluid media. However, in order to achieve the aforementioned gas exchange, a separate gas permeable membrane or gas exchange system would be required.

Chamber 20 may be made of any suitable material (such as a suitable plastic material) and structure to achieve the desired function, in particular to support vascular construct 22 and to provide first and second connections 34 and 36. Vascular construct 22 can be a synthetic structure designed to mimic a human artery (such as a tissue-engineered structure), or may be an explant. One example is Formlabs Biomed Clear Resin (MPN: RS-F2-BMCL-01). Suitable conduits for fluid flow circuit 30 include Masterflex Transfer Tubing (MPN: HV-95666-05) that is ⅛″ ID× 3/16″ OD and of Tygon ND-100-65 material. Suitable conduits for gas circuit 60 include Masterflex Transfer Tubing (MPN: HV-95666-14) that is ¼″ ID×⅜″ OD and of Tygon ND-100-65 material. Suitable tubing connectors that may be used include polypropylene Qosina T connectors (MPN: 61407). Further, it will be appreciated that alternative fluid flow and gas circuits can be used to the configurations depicted in the figures without departing from the invention.

It will be understood that the invention disclosed and defined in this specification extends to all alternative combinations of two or more of the individual features mentioned or evident from the text or drawings. All of these different combinations constitute various alternative aspects of the invention.

1. A system for pumping a fluid through a target unit, comprising:
a source of compressed gas;
a gas circuit connected to said source of compressed gas and including gas flow control apparatus;
a first connection and a second connection for connecting to, respectively, afferent and efferent ends of the target unit;
a fluid flow circuit in fluid communication with said first and second connections, the fluid flow circuit including fluid control apparatus and a first and second fluid reservoir;
wherein the gas flow control apparatus provides coordinated delivery and release of compressed gas to dynamically control fluid level in each of said first and second fluid reservoirs in order to provide, by way of operation of the fluid control apparatus in the fluid flow circuit, continuous fluid flow through the target unit in a single direction in accordance with a predetermined flow profile.

2. The system of claim 1, wherein the fluid control apparatus, alone or in combination with the gas flow control apparatus, allows adjustment of flow and pressure through the target unit.

3. The system of claim 2, wherein the gas flow control apparatus is configured to enable selective and independent adjustment of flow and pressure in the target unit.

4. The system of any one of the preceding claims, wherein the predetermined flow profile is a pulsatile fluid flow designed to mimic hydrodynamic fluid conditions in the vasculature of an animal body.

5. The system of claim 4, further configured to generate and modulate physiological vascular pressure, flow and shear conditions in the pulsatile flow profile in the target unit.

6. The system of any one of the preceding claims, wherein the gas flow control apparatus is configured to adjust a pressure differential between the first reservoir and the second reservoir.

7. The system of claim 6, wherein the gas flow control apparatus is configured to adjust the flow rate of the fluid flow through the target unit by adjusting the pressure differential between the first reservoir and the second reservoir.

8. The system of claim 6 or claim 7, wherein the gas flow control apparatus is configured to adjust a pressure at the target unit by adjusting pressure at the first reservoir and/or the second reservoir.

9. The system of any one of the preceding claims, wherein the gas flow control apparatus comprises a valve arrangement governing provision of the compressed gas to each of the first and the second reservoirs.

10. The system of claim 9, wherein the valve arrangement includes a first proportional solenoid valve controlling connection from the source of compressed gas to the first reservoir and a second proportional solenoid valve controlling connection from the source of compressed gas to the second reservoir.

11. The system of any one of the preceding claims, wherein the gas circuit includes an outlet gas flow apparatus to allow release of the compressed gas on deflation of either of the first and second fluid reservoirs.

12. The system of any one of the preceding claims, wherein the fluid flow circuit is a two-phase recirculation system by which the first connection and the second connection are in fluid communication with the first and second fluid reservoirs under control of said fluid control apparatus.

13. The system of any one of the preceding claims, wherein the fluid control apparatus includes a plurality of one-way valves configured to allow two modes of fluid flow, being (a) driving fluid from the first fluid reservoir to said first connection and from said second connection to the second fluid reservoir, and (b) driving fluid from the second fluid reservoir to said first connection and from said second connection to the first fluid reservoir, alternation between modes (a) and (b) providing the predetermined flow profile.

14. The system of claim 13, wherein the plurality of one-way valves are provided by a passive flow control arrangement, in which the one-way valves are check valves arranged to cause or permit the desired mode of fluid flow in accordance with the operation of the first and second fluid reservoir, or
wherein the plurality of one-way valves are provided by an active flow control arrangement, in which the one-way valves are actively controllable to cause or permit the desired mode of fluid flow.

15. The system of claim 13 or claim 14, wherein coordinated operation of the gas flow control apparatus and the fluid control apparatus enables switching between the two modes of fluid flow.

16. The system of any one of the preceding claims, wherein the system includes the target unit itself, and/or a support for the target unit.

17. The system of claim 16, wherein the support is a housing comprising an afferent port and efferent port, wherein the housing defines a chamber containing the target unit, in which conditions of an interior of the chamber are controlled in order to mimic physiological conditions external to the target unit.

18. A method for pumping a fluid through a target unit in a fluid flow circuit, the fluid flow circuit including two fluid reservoirs and a fluid control apparatus, the method comprising operating gas flow control apparatus to control supply of compressed gas to dynamically control filling and discharge of a first and a second fluid reservoir and, in a manner coordinated with the operation of the gas flow control apparatus, using the fluid control apparatus in the fluid flow circuit to provide fluid flow through the target unit in a single direction in accordance with a predetermined flow profile.

19. The method of claim 18, wherein the method further includes using the fluid control apparatus to alternate connection between two fluid flow routes, namely: (a) from the first fluid reservoir to an afferent end of the target unit and from an efferent end of the target unit to the second fluid reservoir: and (b) from the second fluid reservoir to an afferent end of the target unit and from an efferent end of the target unit to the first fluid reservoir, the alternation between routes (a) and (b) being conducted in accordance with operation of the gas flow control apparatus controlling the operation of the first and second fluid flow reservoirs.

20. The method of claim 18, further including using the gas flow control apparatus to selectively and independently adjust flow and pressure in the target unit.
